(19) United States
(12) Patent Application Publication
(10) Pub. No.: US 2007/0140989 A1
(43) Pub. Date: Jun. 21, 2007

(54) Protein with activity of hydrolyzing dextran, starch, mutan, inulin and levan, gene encoding the same, cell expressing the same, and production method thereof

(76) Inventors: Do-Man Kim, Gwangju (KR); Hee-Kyoung Kang, Gwangju (KR); Jin-Ha Lee, Gwangju (KR)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/588,140

(22) PCT Filed: Jan. 27, 2005
(86) PCT No.: PCT/KR05/00234

§ 371 Date: Jul. 31, 2006

(30) Foreign Application Priority Data
Jan. 30, 2004 (KR) 10-2004-0006185

(51) Int. Cl. A61K 8/96; C07H 21/04; C12P 21/06; C12N 9/32; C12N 15/74; C12N 1/21

(52) U.S. Cl. 424/50; 435/204; 435/69.1; 435/252.33; 435/488; 536/23.2

(57) ABSTRACT

Disclosed is an enzyme, having the amino acid sequence of SEQ. ID. No. 1 with the activity of hydrolyzing dextran, starch, mutan, inulin and levan, a gene encoding the enzyme, and a transformed cell expressing the gene. Also disclosed is a method of producing an enzyme capable of degrading dextran, starch, mutan, inulin and levan, which comprises culturing the cell, expressing the enzyme in the cell and purifying the enzyme. A composition comprising the enzyme is provided for removing dextran or polysaccharide contaminants during sugar production. With such degradation activity, the enzyme not only finds various applications in the dental care industry, including anti-plaque compositions and mouthwashes, but is also useful in removing dextran or polysaccharide contaminants during sugar production.

### BACKGROUND OF THE INVENTION

1. Field of the Invention

The present invention relates to an enzyme capable of hydrolyzing dextran, starch, mutan, inulin and levan, a gene thereof, an expression cell thereof, and a production method thereof. More particularly, the present invention relates to an enzyme useful not only in anti-plaque compositions or mouthwashes due to its ability to inhibit the formation of dental plaque and degrade previously formed plaque, but also in dextran removal during sugar production due to its excellent ability to hydrolyze dextran, a gene coding for the enzyme, a cell expressing the enzyme, and a method of producing the enzyme.

2. Description of the Related Art

Plaque is a biofilm built up on the teeth, resulting from microbial colonization of the tooth surface. The bulk of dental plaque is composed of bacteria-derived extracellular polysaccharide known as glucan (insoluble glucan), also called mutan, which enhances the colonization. Amounting to about 20% of the dried weight of plaque, this polysaccharide acts as an important factor to cause dental caries. Structural studies of glucans produced by Streptococcus mutans revealed that glucose moieties of the insoluble glucans are linked to each other mainly via α-1,3-, α-1,4-, and α-1,6-D-glucosidic bonds. Effective elimination of plaque, therefore, demands mutanolytic, amylolytic and dextranolytic activities.

Conventionally, the prevention of the formation of plaque and dental caries has mainly depended on the inhibition of the growth of Streptococcus mutans (S. mutans) in the mouth. In this regard, compounds with activity against S. mutans growth, such as antiseptics or fluorine, are included in oral products such as toothpastes or mouthwash. Inhibitory as it is of the growth of S. mutans, fluorine, which is a popular anti-tooth cavity compound, gives rise to dental fluorosis (formation of mottles in the dental enamel) as well as causing side effects such as strong toxicity and air pollution. Another attempt has been made to prevent dental caries with enzymes such as dextranase; however, its effect has yet to be proven.

U.S. Pat. No. 5,741,773 provides a dentifrice composition containing glycomacropeptide having antiplaque and anticaries activity. This conventional technique is directed to inhibiting the growth of the bacteria that cause dental caries. However, nowhere are suggested the prevention of plaque formation or the hydrolysis of previously formed plaque.

U.S. Pat. No. 6,485,953 (corresponding to Korean Pat. No. 10-0358376), issued to the present inventors, suggests the use of DXAMase capable of hydrolyzing polysaccharides of various structures in inhibiting the formation of dental plaque and degrading previously formed dental plaque. In addition to an enzyme capable of degrading various polysaccharides, a microorganism (Lipomyces starkeyi KFCC-11077) producing the enzyme and a composition containing the enzyme are also disclosed.

However, there continues to be a demand for a novel enzyme which can more effectively inhibit the formation of plaque and hydrolyze previously formed plaque.

In Korean Pat. Appl'n No. 10-2001-48442, the present inventors also suggested that the enzyme DXAMase produced by the microorganism (Lipomyces starkeyi KFCC-11077) of Korean Pat. No. 10-0358376 can be useful in removing dextran due to its high dextran-degrading activity.

There is therefore a clear need in the art to develop a new enzyme having dextran degradation activity sufficient for dextran removal during sugar production.

### SUMMARY OF THE INVENTION

Accordingly, the present invention has been made keeping in mind the above problems occurring in the prior art, and an object of the present invention is to provide a novel enzyme having the activity of preventing plaque formation and degrading previously formed dental plaque as well as excellent dextranolytic activity, and a gene encoding the enzyme.

It is another object of the present invention to provide a strain which carries the gene.

It is a further object of the present invention to provide a method of producing the enzyme and the gene.

It is still a further object of the present invention to provide an industrially useful composition comprising the enzyme.

In accordance with an aspect of the present invention, there are provided a protein, comprising an amino acid sequence of SEQ. ID. No. 1, which has the activity of hydrolyzing dextran, starch, mutan, inulin and levan, a derivative thereof, or a fragment thereof.

In accordance with another aspect of the present invention, there is provided a gene of SEQ. ID. No. 2, encoding the protein, the derivative or the fragment, a derivative thereof, or a fragment thereof.

In accordance with a further aspect of the present invention, there is provided a transformed cell, expressing the gene.

In accordance with still a further aspect of the present invention, there is provided a method of producing an enzyme having activity of hydrolyzing dextran, starch, mutan, inulin and levan, comprising: culturing the cell; expressing the enzyme in the cultured cell; and purifying the expressed enzyme.

### BRIEF DESCRIPTION OF THE DRAWINGS

The above and other objects, features and advantages of the present invention will be more clearly understood from the following detailed description taken in conjunction with the accompanying drawings, in which:

FIG. 1 shows an amino acid sequence of the carbohydrolase derived from Lipomyces starkeyi (LSD1) according to the present invention and a 2052 bp nucleotide sequence encoding the amino acid sequence, wherein PCR primers for cloning the protein in a vector are underlined;

FIG. 2 is a graph in which the activity and stability of the LSD of the present invention are plotted versus pH value;

FIG. 3 is a graph in which the activity and stability of the LSD of the present invention are plotted versus temperature;

FIG. 4 is a photograph of a TLC result showing the enzymatic activity of the LSD of the present invention before and after carrying out enzyme deactivation (lanes 1 to 5 and lanes 6 to 10, respectively, in which samples of starch (lanes 1 and 6), dextran (lanes 2 and 7), mutan (lanes 3 and 8), levan (4 and 9) and inulin (lanes 5 and 10) are analyzed, along with a series of maltodextrins (lane Mn) and a series of isomaltodexrins (lane IMn) after the enzyme extract is allowed to react with the samples; and

FIG. 5 is a graph showing the binding ability of the enzyme of the present invention to hydroxyapatite, along with that of Penicillium dextranase.

### DESCRIPTION OF THE PREFERRED EMBODIMENTS

The acquisition of a gene coding for the carbohydrolase (i.e. glycanase(LSD)) of the present invention starts by culturing Lipomyces starkeyi in a medium containing dextran and isolating poly(A)+ RNA from the microorganism. Next, on the basis of the information about the amino acid sequences which are common in the genes coding for dextranases known thus far, primers comprising expected conserved regions are constructed, followed by PCR with the primers. The PCR product, approximately 1.1 kb long, is used for 5′ RACE and 3′ RACE to allow for a complete dextranase gene. After being amplified by PCR, the gene is cloned in the Saccharomyces cerevisiae vector pYES2 with which transformation is carried out in S. cerevisiae. The cells which have undergone the transformation are grown in a medium containing blue dextran and galactose. Colonies around which a clear halo is formed against the blue background are selected (S. cerevisiae INVSc1) and from the S. cerevisiae transformant, a recombinant clone carrying the gene of interest is obtained (pYLSD1).

L. starkeyi is known to produce endo-dextranase (EC 3.2.1.11), which degrades dextran, and α-amylase which degrades starch. This microorganism has been applied to foods and has not yet been reported to produce antibiotics or other toxic metabolites.

Most of the dextranases produced by microorganisms, except for a few derived from bacteria, are known as inducible enzymes. L. starkeyi ATCC 74054, first reported in U.S. Pat. No. 5,229,277, produces both dextranase and amylase whose characteristics are also disclosed. It has also been reported that the strain produces low molecular weight dextrans from sucrose and starch. On the basis of the findings, the present inventors acquired Korean Pat. No. 10-0358376 on Oct. 11, 2002 (corresponding to U.S. Pat. No. 6,485,953 dated Nov. 26, 2002) which relates to a DXAMase enzyme capable of hydrolyzing both dextran and starch, a microorganism producing the enzyme (identified as Lipomyces starkeyi KFCC-11077), and a composition comprising the enzyme.

The enzyme expressed from the gene (lsd1) of the present invention is capable of hydrolyzing starch and mutan (insoluble glucan) as well as dextran. Also, the glycanase according to the present invention is found to degrade dextran mainly into glucose, isomaltose and isomaltotriose, with the concurrent production of smaller amounts of branched pentaoses and hexaoses.

Both levan- and inulin-type fructans, which are constituents of dental plaque, can be degraded by the glycanase according to the present invention.

Accordingly, effective degradation of glucans, whether soluble or insoluble, can be achieved by the glycanase of the present invention. As it can prevent the formation of plaque and remove previously formed plaque by inhibiting the colonization of bacteria and the aggregation of glucans, the glycanase is useful in preventing tooth cavities. It is inferred that the glycanase has the ability to remain on the teeth as demonstrated by a test for whether or not the enzyme binds to hydroxyapatite which is similar to tooth enamel components.

Also, the present invention is concerned with a novel microorganism carrying a gene encoding the glycanase. The microorganism, a Saccharomyces cerevisiae strain, was deposited in the Korean Collection for Type Cultures (KCTC) located in Yusung Gu, Daejeon City, South Korea, with the accession number KCTC10574BP, on Dec. 24, 2003.

Also, the present invention pertains to a method of producing the glycanase. First, the clone pYLSD1 is amplified by cell culture. After being harvested from the culture, the cells are disrupted using glass beads to isolate the glycanase therefrom. The glycanase encoded by pYLSD1 is substantially identical in characteristics to that of L. starkeyi KFCC-11077.

Lipomyces starkeyi KFCC 11077, used as a DNA donor for RNA isolation and glycanase gene selection, produces glycanase which has dextranase and amylase activity.

To provide DNA of interest, Lipomyces starkeyi KFCC 11077 is aerobically cultured in an LMD medium containing 1% (w/v) dextran, a 1% (v/v) mineral solution and 0.3% (w/v) yeast extract. The mineral solution contains 2% (w/v) MgSO₄.7H₂O, 0.1% (w/v) NaCl, 0.1% (w/v) FeSO₄.7H₂O, 0.1% (w/v) MnSO₄.H₂O, and 0.13% (w/v) CaCl₂.2H₂O. In the present invention, general DNA manipulation and DNA sequencing are carried out with Escherichia coli DH5α and pGEM-T easy (Promega, USA).

As a host cell for pYLSD1, S. cerevisiae INVSc1 is cultured in an YPD medium (yeast extract 10 g/l, peptone 20 g/l and glucose 20 g/l) so as to express the glycanase. The YPD medium for S. cerevisiae culture is supplemented with synthetic dextrose (SD) and a synthetic complement.

A composition comprising the enzyme of the present invention may be used in a variety of oral care applications, including anti-plaque compositions, mouthwashes, toothpastes, etc. By virtue of its ability to degrade polysaccharides such as dextran and starch, the enzyme of the present invention is also effectively used to remove dextran during sugar production. Additionally, compositions comprising the enzyme according to the present invention can be applied to foods such as gum, drinks, milks, etc. and their constituents may be readily determined by those who are skilled in the art.

A better understanding of the present invention may be obtained through the following examples which are set forth to illustrate, but are not to be construed as the limit of the present invention.

### EXAMPLE 1

### Isolation of Poly A+ RNA from L. starkeyi

L. starkeyi was inoculated into an LMD medium. After being culturing at 28° C. for 36 hours (to the mid-exponential growth phase), the culture was centrifuged at 6,500×g to form a cell pellet. This pellet was suspended in a GIT buffer (4M guanidine isothiocitrate, 25 mM sodium citrate (pH 7.0), 0.5% lauroylsarcosyl in 0.1% DEPC-treated water, 0.1M 2-mercaptoethanol) and mixed with acid-washed glass beads and an equal volume of phenol (pH 4.0). After voltexing the mixture for 2 min, centrifugation was conducted. Addition of isopropanol to the supernatant gave rise to the precipitation of total RNA. By using oligotex resins (Oligotex mRNA kit, Quiagen) to form an oligotex-mRNA complex, mRNA was purified from the total RNA preparation.

### EXAMPLE 2

### RT-PCT Amplification of LSD1

For the first strand CDNA synthesis, reverse transcription was conducted with 0.5 g of the total RNA isolated from L. starkeyi, in the presence of the modified oligo-dT primer T18NN (5′-GAGAGAGAGAGAGAGAGAGAACTAGTCTCGAGTTTTTTTTTTTTTTTTTT-3′). 10 μl of the first strand cDNA was used to amplify a part of the base sequence coding for glycanase. A pair of the degenerated primers DC-F and DC-R was constructed with reference to seven conserved regions known in dextranase. The design of the primers DC-F (5′-ACCTGGCA(T/C)AG(A/G) (A/T/G) (A/C) (C/A)-3′) and DC-R (5′-G(G/C) (C/T) (T/G)CC(G/C)ACCTGCTT(A/G)TA-3′) was based on the peptide sequences TWWH(D/N) (N/S/T) (conserved region I) and YKQVG(S/A) (conserved region V), respectively. Using these primer sets, PCR was conducted to give a putative glycanase gene fragment of about 1.1 kb. The PCR product was purified from the agarose gel with the help of an AccPrep™ gel extraction kit (Bioneer, Korea), followed by the ligation of the purified DNA fragment to a pGEM-T easy vector (Promega, USA). DNA sequencing was conducted in the Korea Basic Science Institute. To obtain an intact gene for glycanase, RACE (rapid amplification of CDNA ends) was carried out on the basis of the information about the 1.1 kb DNA fragment. In this regard, 5′-RACE and 3′-RACE depended on 5′-full RACE Core Set and 3′-full RACE Core Set (both TaKaRa, Japan) so as to allow for a full size cDNA encoding glycanase. Through the 5′-RACE, a 180 bp PCR product was obtained while the 3′-PACE resulted in a 900 bp PCR product. Therefore, a glycanase gene (lsd1), about 2 kb long in total, was acquired.

### EXAMPLE 3

### Base and Amino Acid Sequencing of Glycanase Gene

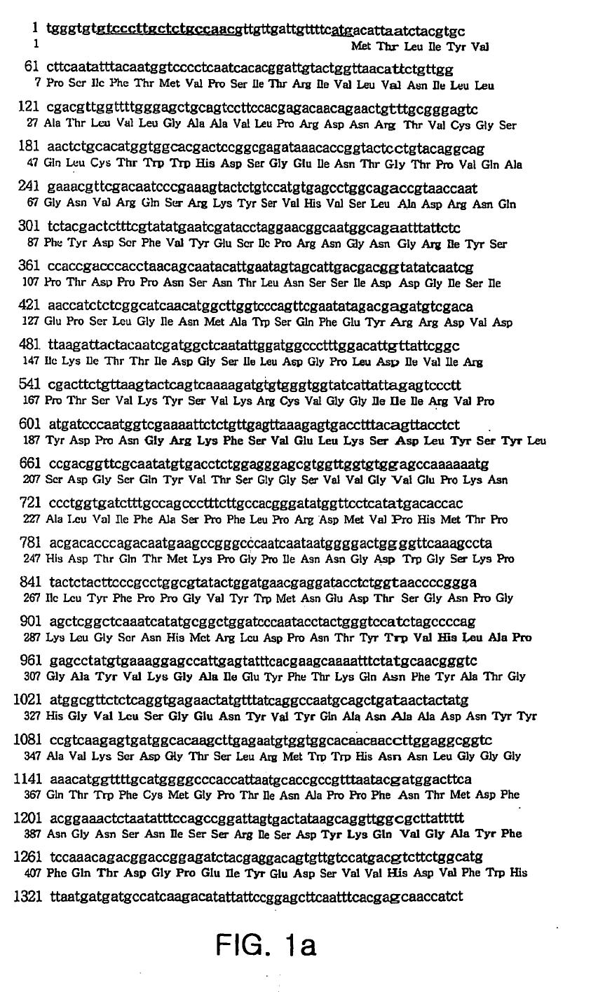

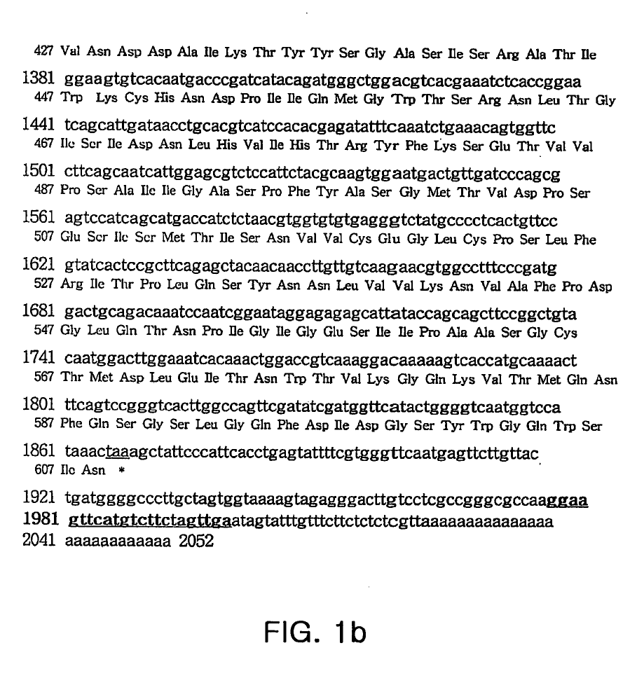

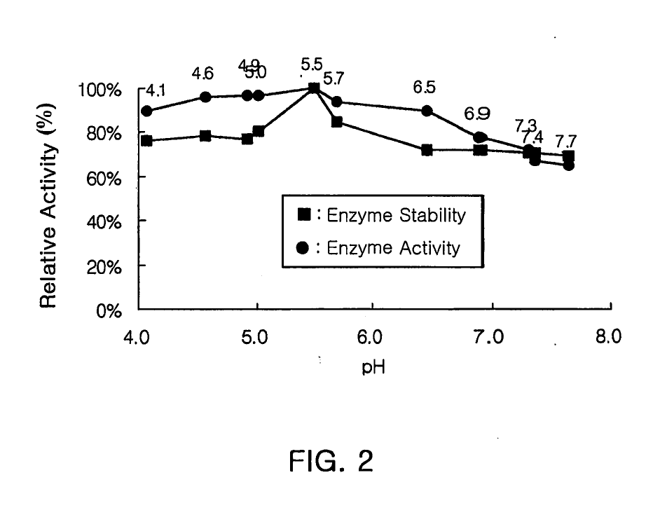

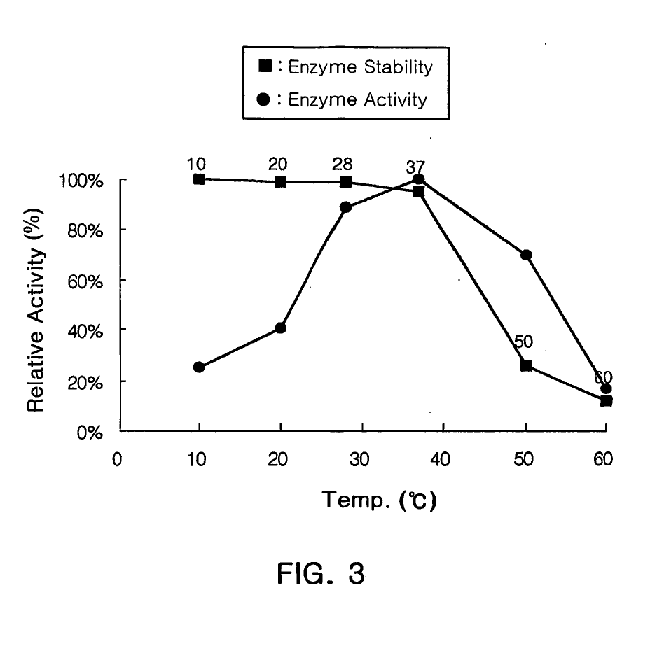

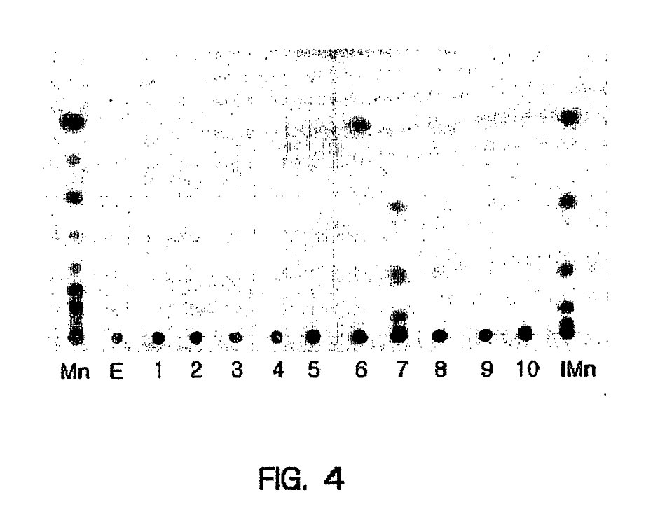

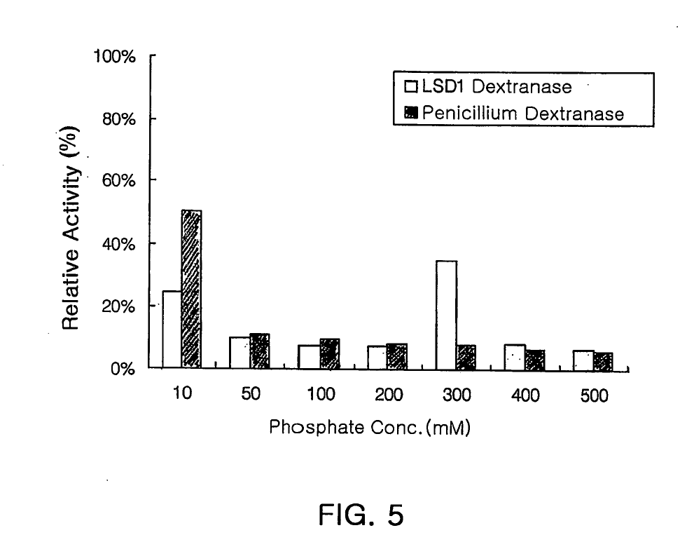

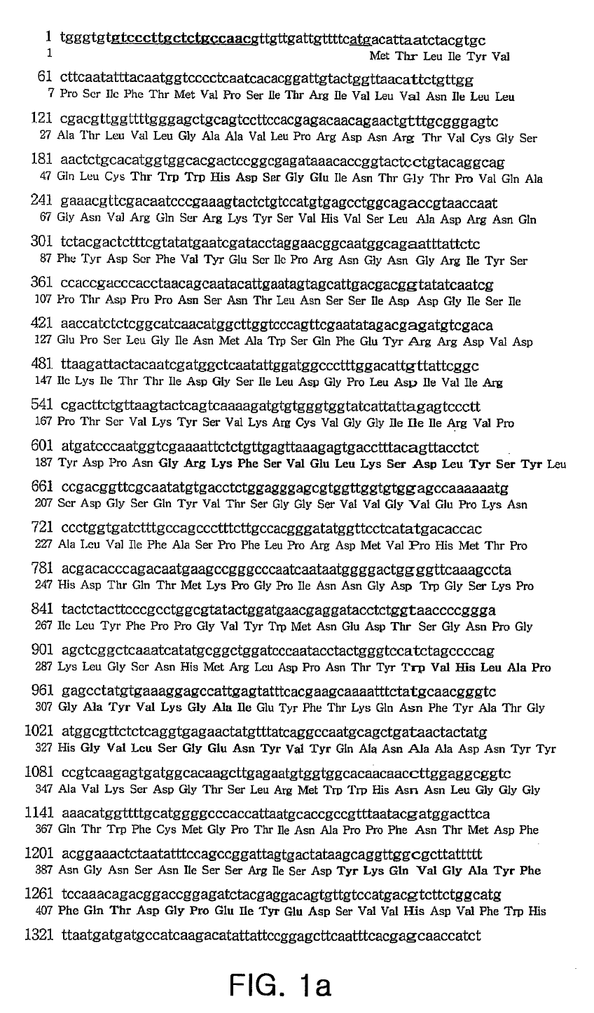

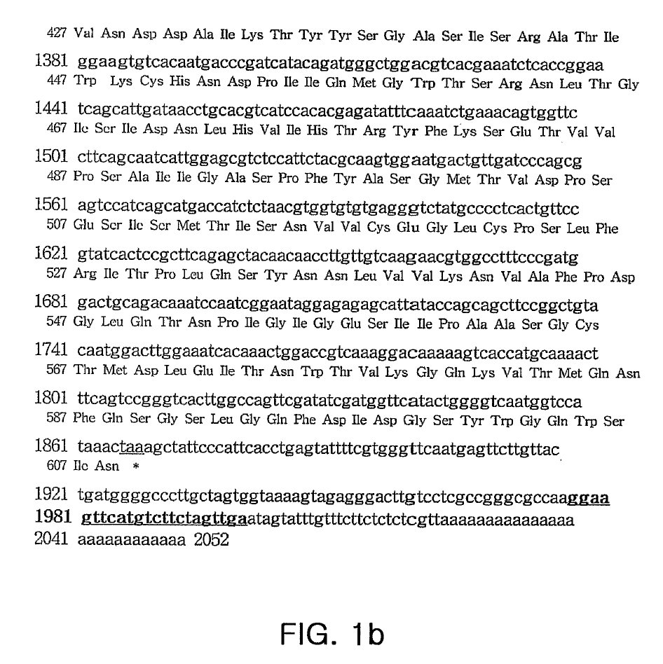

By an alkaline lysis method, a plasmid DNA was prepared for base sequencing. With the aid of ABI PRISM Cycle Sequencing Kit (Perkin Elmer Corp. USA), base sequencing was performed in a GeneAmP 9600 thermal cycler DNA sequencing system (Model 373-18, Applied Biosystems, USA). The base sequencing result is given in FIG. 1 and SEQ. ID. Nos. 1 and 2.

The DNA fragment containing a glycanase gene was found to have an open reading frame consisting of 1824 base pairs. The open reading frame starts with the initiation codon (ATG) at nucleotide position 42 of the acquired base sequence and terminates with the stop codon (TGA) at nucleotide position 1,868. Consisting of 608 amino acid residues, the putative protein corresponding to the structural gene was calculated to have a molecular weight of 67.6 kDa.

### EXAMPLE 4

### Construction of Recombinant Plasmid pYLSD1 and Transformation of S. cerevisiae Therewith

L. starkeyi was cultured in YPD and harvested, and genomic DNA was isolated according to the Schwartz and Cantor method.

Using a set of the synthetic primers DX-F: 5′-GTCCCTTGAGCTCCCAAC-3′ (Sequence List 3) and DX-R: 5′-TCAACTAGAATTCATGAACTTCC-3′ (Sequence List 4), PCR was carried out in the presence of Taq DNA polymerase with 30 cycles of denaturing temperature at 94° C. for 1 min, annealing temperature at 52° C. for 1 min and extending temperature at 72° C. for 2 min while a DNA fragment corresponding to the glycanase gene (lsd1) served as a template. The PCR product was ligated with a PGEM-T easy vector with which transformation was carried out. The plasmid prepared from the transformed cells was treated with EcoRI to excise the PCR product which was then ligated with a pYES2 vector (Invitrogen, USA). In this regard, the vector was previously digested with EcoRI and treated with CIAP for preventing self-ligation. The transfection of the resulting recombinant plasmid into S. cerevisiae was carried out with an electroporation method. Selection for transformants grown in an SC medium utilized an induction medium (2% galactose, 0.3% blue dextran, lacking uracil). When SC plates inoculated with the transformants were incubated at 30° C. for two to six days, halos resulting from the hydrolysis of dextran were formed around colonies if they anchored the recombinant plasmid. Colonies around which a clear halo was formed against the blue background were selected and the clone carrying the gene of interest was named pYLSD1.

### EXAMPLE 5

### Selection of Bacteria Expressing Glycanase Gene

Galactose induction was conducted to examine the activity of the clone in a supernatant. The selected colonies were inoculated into 50 ml of an SC liquid medium containing 2% galactose and 1% glucose, in such an amount as to reach OD600=1, followed by incubation at 30° C. for 72 hours Cells were harvested by centrifugation (5,000 rpm×5 min) and suspended in 5 ml of a 20 mM citrate/phosphate buffer (pH 5.5), after which cell disruption was conducted by vortexing for 3 min in the presence of 0.1 g of 0.45 mm glass beads. The cell lysate was centrifuged at 6,000 rpm for 2 min, after which the supernatant was carefully recovered. The supernatant was reacted with polyethylene glycol (PEG, Mw=150,000-200,000) at 4° C. to a concentrated volume to remove glucose, disaccharides and oligosaccharides therefrom. The PEG concentrate was dialyzed against 20 mM citrate/phosphate buffer (pH 5.5) to the original volume. Serving as a crude enzyme extract for determining protein activity, the dialyzate solution was mixed with an equal volume of 1% dextranase. 16 hours after reaction, the activity was measured.

### EXAMPLE 6

### Assay for Enzyme Activity

The reducing value of the enzyme was determined by a copper-bicinchoninate method. That is, 100 μl of copper-bicinchoninate was added to 100 μl of an enzyme solution, and allowed to react at 80° C. for 35 min, followed by being cooled for about 15 min. Absorbance was measured at 560 mm. The dextranase activity of the glycanase enzyme was determined by measuring the amount of isomaltose produced when the crude enzyme extract was allowed to react with 2% dextran buffer at 37° C. for 15 min. A unit of dextranase activity is defined as the amount of enzyme which produces 1 μmol of isomaltose when reacting with dextran at 37° C. for 1 min.

### EXAMPLE 7

### Assay for Optimal pH and Temperature and Stability of Glycanase

The dextranase activity of the glycanase was assayed for optimal pH by measuring the dextranase activity in the range of pH 4.1-7.7 after the reaction of the enzyme with dextran for 16 hours. The stability of the enzyme to pH was determined after the enzyme was allowed to stand for 3 hours at 22° C. in each buffer.

The optimal temperature of the enzyme was determined by measuring the reaction rates of the enzyme which had been allowed to stand for 16 hours at various temperatures (10-60° C.). For the determination of temperature stability, the enzyme was measured for residual activity after being allowed to stand for 3 hours at various temperatures (10-60° C.).

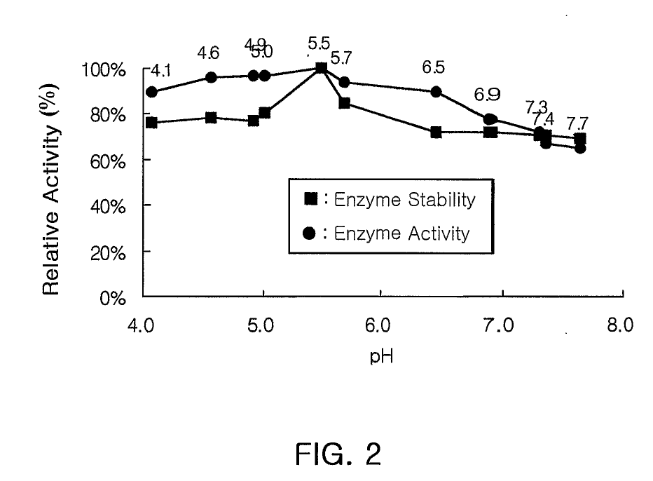

The LSD enzyme was found to show optimal dextranase activity at pH 5.5 and maintain 80% or more of the optimal activity at pH 5.0-5.7 (FIG. 2, Table 1).

TABLE 1
pH Effect on Glycanase Activity and Stability

| | Dextranase Activity |
|---|---|
| Optimal pH | 5.5 |
| Stable pH range | 5.0-5.7 |

In Table 1, the stable pH means that the residual activity of the enzyme is 80% or more of the initial activity at that pH range.

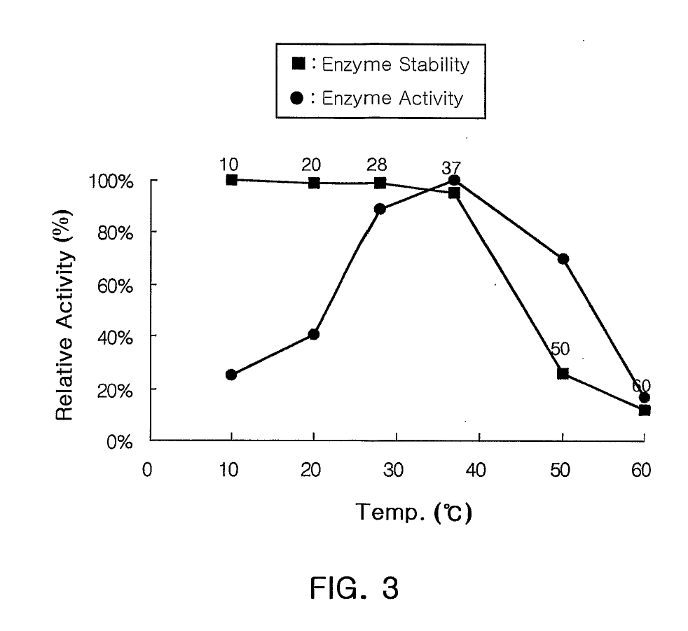

Also, the enzyme showed 80% or more of the initial activity at temperatures less than 37° C., with the optimal activity at 37° C. (FIG. 3, Table 2).

TABLE 2
Effect of Temperature on Glycanase Stability

| | Dextranase Activity |
|---|---|
| Stable Temp. range | ≦37° C. |

In Table 2, the stable temperature range means that the residual activity of the enzyme is 80% or more of the initial activity in that temperature range.

### EXAMPLE 8

### Degradation Activity of Dextranase for Various Substrates

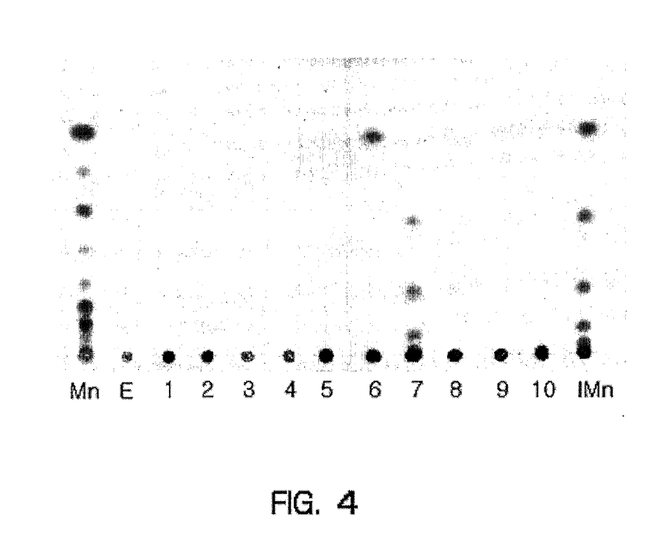

The crude enzyme extract was examined for degradation activity for various substrates (FIG. 4). In addition to glucan, 1% aqueous solutions of various polymers, including dextran, starch, levan (β-2,6 linked D-fructose polymer), inulin (β-2 μl linked D-fructose polymer), mutan and (α-1,3 linked D-glucose polymer) were prepared for hydrolysis activity test. The reaction of the enzyme with dextran resulted in 0.1% glucose, 19.3% isomaltose, 24.2% isomaltotriose and 17.0% isomaltotetraose, with the concurrent production of branched oligosaccharides. Therefore, the glycanase is believed to act as an endo-dextranase in the reaction with dextran. In the presence of the glycanase, starch was found to be almost completely degraded into glucose.

In addition, the glycanase expressed from the clone pYLDS1 was assayed for hydrolysis activity through the reaction with various polymers. Low as it was, the hydrolysis activity of the glycanase was detected not only with α-1,3-D-glucoside linked polymers such as mutan but also with β-linked polymers such as inulin. The glycanase was measured to have a hydrolysis activity of 54% for starch, 8% for mutan, 3% for levan, and 7% for inulin relative to 100% for dextran.

TABLE 3
Relative Activity of Glycanase for Various Substrates

Relative Activity (%)

| | Glycanase of Mother Cell | LSD1 |
|---|---|---|
| Substrates | (L. starkeyi) | Glycanase |
| Dextran | 100 | 100 |
| Starch | 92 | 54 |
| Mutan | 16 | 8 |
| Levan | 22 | 3 |
| Inulin | 18 | 7 |

### EXAMPLE 9

### Binding of Dextranase to Hydroxyapatite (HA)

By virtue of their ability to bind directly to bone, calcium phosphate ceramics are popularly used as substitutes for bone. Of them, hydroxyapatite (HA) is the most suitable for study on artificial bones and teeth because it shows crystallographic properties similar to those of naturally occurring apatite found in the tooth. For this reason, HA was adopted as a material for testing the bonding ability of the enzyme to the teeth. HA (Bio-Gel HTP, Bio-Rad Laboratories, Richmond Calif.) was suspended in a 10 mM phosphate buffer (pH 6.8). Separately, the enzyme was also suspended in the same buffer. 200 μl of the HA suspension was mixed with an equal volume of the enzyme suspension, after which the mixture was allowed to stand for 60 min for the HA to adsorb enzyme thereonto. After the enzymes remaining free were washed out, elution was conducted with 10, 50, 100, 200, 300, 400 and 500 mM phosphate buffers (pH 6.8), each containing 1 mM NaCl. After being collected, enzyme eluted fractions were assayed for glycanase activity.

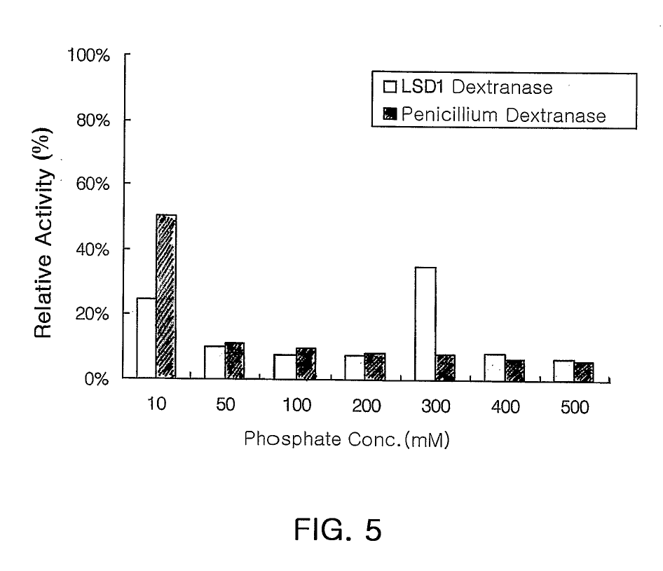

As seen in FIG. 5, the glycanase was eluted with 300 mM hydroxyapatite. It is also understood that the remnant of the glycanase in apatite is higher than that of Penicillium dextranase. Taken together, these results reveal that the glycanase strongly binds to hydroxyapatite and thus can stay in the teeth.

As described hereinbefore, the glycanase produced from the Lipomyces starkeyi mutant of the present invention is a single protein of about 70 kDa, which is found to have an open reading frame consisting of 1,824 bp nucleotides as analyzed by base sequencing with the PCR product thereof. The putative protein of the structural gene consists of 608 amino acid residues with a molecular weight of about 67.6 kDa.

The final products resulting from the reaction of glycanase with dextran are nothing but the typical products of endo-dextranase. The enzyme of interest degrades dextran mainly into glucose, isomaltose, isomaltotriose and isomaltotetraose, with the concurrent production of branched pentasaccharides. Additionally, the enzyme is found to exert degradation activity to a variety of carbohydrates, including α-1,3-D-glucoside linked polymers as well as β-linked fructan such as levan and inulin.

With the above-mentioned degradation activity, therefore, the enzyme of the present invention not only finds various applications in the dental care industry, including anti-plaque compositions and mouthwashes, but is also useful in removing dextran or polysaccharide contaminants during sugar production.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A protein, comprising an amino acid sequence of SEQ. ID. No. 1, which has the activity of hydrolyzing dextran, starch, mutan, inulin and levan, a derivative thereof, or a fragment thereof.

2. A gene of SEQ. ID. No. 2, encoding the protein, the derivative, or the fragment of claim 1, a derivative thereof, or a fragment thereof.

3. A transformed cell, expressing the gene, the derivative, or the fragment of claim 2.

4. The transformed cell as defined in claim 3, wherein the cell is prokaryotic or eukaryotic.

5. The transformed cell as defined in claim 4, wherein the cell is Saccharomyces cerevisiae pYLSD1 deposited on Dec. 24, 2003, with the accession number KCTC 10574BP.

6. A method of producing an enzyme having activity of hydrolyzing dextran, starch, mutan, inulin and levan, comprising:
culturing the cell of claim 3;
expressing the enzyme in the cultured cell; and
purifying the expressed enzyme.

7. An enzyme, produced by the method of claim 6.

8. A composition, comprising the enzyme of claim 7.

9. The composition as defined in claim 8, wherein the composition is used for dextran removal during sugar production.

10. The composition as defined in claim 8, wherein the composition is used for plaque elimination or as a mouthwash.


SEQUENCE LISTING (4 sequences)

SEQ ID NO: 1 - protein, 608 aa, Artificial Sequence
  S. cerevisiae/pYES2-LSD1
MTLIYVPSIFTMVPSITRIVLVNILLATLVLGAAVLPRDNRTVCGSQLCTWWHDSGEINTGTPVQAGNVRQSRKYSVHVSLADRNQFYDSFVYESIPRNGNGRIYSPTDPPNSNTLNSSIDDGISIEPSLGINMAWSQFEYRRDVDIKITTIDGSILDGPLDIVIRPTSVKYSVKRCVGGIIIRVPYDPNGRKFSVELKSDLYSYLSDGSQYVTSGGSVVGVEPKNALVIFASPFLPRDMVPHMTPHDTQTMKPGPINNGDWGSKPILYFPPGVYWMNEDTSGNPGKLGSNHMRLDPNTYWVHLAPGAYVKGAIEYFTKQNFYATGHGVLSGENYVYQANAADNYYAVKSDGTSLRMWWHNNLGGGQTWFCMGPTINAPPFNTMDFNGNSNISSRISDYKQVGAYFFQTDGPEIYEDSVVHDVFWHVNDDAIKTYYSGASISRATIWKCHNDPIIQMGWTSRNLTGISIDNLHVIHTRYFKSETVVPSAIIGASPFYASGMTVDPSESISMTISNVVCEGLCPSLFRITPLQSYNNLVVKNVAFPDGLQTNPIGIGESIIPAASGCTMDLEITNWTVKGQKVTMQNFQSGSLGQFDIDGSYWGQWSIN

SEQ ID NO: 2 - DNA, 2052 bp, Artificial Sequence
  S. cerevisiae/pYLSD1
tgggtgtgtcccttgctctgccaacgttgttgattgttttcatgacattaatctacgtgccttcaatatttacaatggtcccctcaatcacacggattgtactggttaacattctgttggcgacgttggttttgggagctgcagtccttccacgagacaacagaactgtttgcgggagtcaactctgcacatggtggcacgactccggcgagataaacaccggtactcctgtacaggcaggaaacgttcgacaatcccgaaagtactctgtccatgtgagcctggcagaccgtaaccaattctacgactctttcgtatatgaatcgatacctaggaacggcaatggcagaatttattctcccaccgacccacctaacagcaatacattgaatagtagcattgacgacggtatatcaatcgaaccatctctcggcatcaacatggcttggtcccagttcgaatatagacgagatgtcgacattaagattactacaatcgatggctcaatattggatggccctttggacattgttattcggccgacttctgttaagtactcagtcaaaagatgtgtgggtggtatcattattagagtcccttatgatcccaatggtcgaaaattctctgttgagttaaagagtgacctttacagttacctctccgacggttcgcaatatgtgacctctggagggagcgtggttggtgtggagccaaaaaatgccctggtgatctttgccagccctttcttgccacgggatatggttcctcatatgacaccacacgacacccagacaatgaagccgggcccaatcaataatggggactggggttcaaagcctatactctacttcccgcctggcgtatactggatgaacgaggatacctctggtaaccccgggaagctcggctcaaatcatatgcggctggatcccaatacctactgggtccatctagccccaggagcctatgtgaaaggagccattgagtatttcacgaagcaaaatttctatgcaacgggtcatggcgttctctcaggtgagaactatgtttatcaggccaatgcagctgataactactatgccgtcaagagtgatggcacaagcttgagaatgtggtggcacaacaaccttggaggcggtcaaacatggttttgcatggggcccaccattaatgcaccgccgtttaatacgatggacttcaacggaaactctaatatttccagccggattagtgactataagcaggttggcgcttattttttccaaacagacggaccggagatctacgaggacagtgttgtccatgacgtcttctggcatgttaatgatgatgccatcaagacatattattccggagcttcaatttcacgagcaaccatctggaagtgtcacaatgacccgatcatacagatgggctggacgtcacgaaatctcaccggaatcagcattgataacctgcacgtcatccacacgagatatttcaaatctgaaacagtggttccttcagcaatcattggagcgtctccattctacgcaagtggaatgactgttgatcccagcgagtccatcagcatgaccatctctaacgtggtgtgtgagggtctatgcccctcactgttccgtatcactccgcttcagagctacaacaaccttgttgtcaagaacgtggcctttcccgatggactgcagacaaatccaatcggaataggagagagcattataccagcagcttccggctgtacaatggacttggaaatcacaaactggaccgtcaaaggacaaaaagtcaccatgcaaaactttcagtccgggtcacttggccagttcgatatcgatggttcatactggggtcaatggtccataaactaaagctattcccattcacctgagtattttcgtgggttcaatgagttcttgttactgatggggcccttgctagtggtaaaagtagagggacttgtcctcgccgggcgccaaggaagttcatgtcttctagttgaatagtatttgtttcttctctctcgttaaaaaaaaaaaaaaaaaaaaaaaaaaa

SEQ ID NO: 3 - DNA, 18 bp, Artificial Sequence
  L. starkeyi DX-F primer(sense)
gtcccttgagctcccaac

SEQ ID NO: 4 - DNA, 23 bp, Artificial Sequence
  L. starkeyi DX-R primer(antisense)
tcaactagaattcatgaacttcc